(19) United States
(12) Patent Application Publication
(10) Pub. No.: US 2022/0096016 A1
(43) Pub. Date: Mar. 31, 2022

(54) STATE ESTIMATION APPARATUS, STATE ESTIMATION METHOD, AND COMPUTER-READABLE RECORDING MEDIUM

(71) Applicant: NEC Corporation, Minato-ku, Tokyo (JP)

(72) Inventor: Masanori TSUJIKAWA, Tokyo (JP)

(73) Assignee: NEC Corporation, Minato-ku, Tokyo (JP)

(21) Appl. No.: 17/427,228

(22) PCT Filed: Feb. 1, 2019
(86) PCT No.: PCT/JP2019/003750

§ 371 Date: Jul. 30, 2021

(51) Int. Cl. A61B 5/00; A61B 5/024

(52) U.S. Cl. CPC A61B 5/7203; A61B 5/024

(57) ABSTRACT

A state estimation apparatus 10 includes: a filter generation unit 11 that generates a filter for emphasizing heart rate component and heart rate variable component by removing noise contained in a time series signal of a pulse wave obtained by an image data of a face image of a human; a noise removal unit 12 that performs a convolutional calculation between the bandpass filter generated by the filter generation unit 11 and the time-series signal of the pulse wave containing noise, thereby obtains a pulse wave after noise removal; a state estimation unit 13 that receives the time-series signal of the pulse wave after noise removal, which is the output of the noise removal unit 12, and outputs a state estimation value with reference to a state estimation model learned in advance.

### TECHNICAL FIELD

The present invention relates to a state estimation apparatus and a state estimation method for estimating a state of human, and further relates to a computer-readable recording medium for realizing the apparatus and the method.

### BACKGROUND ART

With the spread of wearable sensors in recent years, research and development on a technique for estimating a state of a human from biological information, particularly information indicating the movement of the heart such as a heart rate and a pulse (both are called heart rate), is being actively carried out. A time variable of heart rate and pulse (both called heart rate variable) are used as indicators of autonomic nervous activity and it is considered to be useful for estimation of various states of human such as discrimination of sleep state, determination of drowsiness, determination of stress level, determination of emotion, determination of mental workload, determination of diseases affecting autonomic nerves, detection of arrhythmia.

For example, Non-Patent Document 1 describes technology to determine sleep or wakefulness from a heart rate calculated from an ECG signal measured by an electrocardiogram (ECG) or a heart rate calculated from a PPG signal measured by a photoelectric volume pulse wave meter (PPG). The technique disclosed in Non-Patent Document 1 is a technique for automatically extracting a heartrate variable component by a learned neural network to finally determine sleep or wakefulness. Further, in the learning process in Non-Patent Document 1, a time-series signal of the calculated heart rate is input to a convolutional neural network (CNN), and CNN parameters are updated so that the output determination result of sleep or wakefulness matches a correct label (sleep or wakefulness).

### LIST OF RELATED ART DOCUMENTS

### Non-Patent Document

Non-Patent Document 1: John Malik, Yu-Lun Lo, and Hau-tieng Wu, “ Sleep-wake classification via quantifying heart rate variability by convolutional neural network,” Physiological Measurement 39, 085004, 2018.

### SUMMARY OF INVENTION

### Problems to be Solved by the Invention

Incidentally, as described above, according to the technique disclosed in Non-Patent Document 1, it is possible to determine whether a human is sleeping or wakefulness by using the time-series signal of the heart rate, but it is necessary to calculate a hear rate with high time resolution and high accuracy in order to extract the heart rate variable component. Therefore, it is necessary to attach sensors for detecting the movement of the heart (ECG sensor and PPG sensor in Non-Patent Document 1) to each human's skin without any gap. As a result, there is a problem that the human who wears the sensor has a feeling of restraint and feels a burden in wearing the sensor.

On the other hand, there is known a technique for detecting a heart rate from a wave (pulse wave) indicating an operation of the heart obtained by various sensors such as a wristband type wearable sensor with a built-in PPG sensor, a sensor installed on a bed or chair that measures pressure or vibration, a sensor that measures sound, and a sensor for radiating microwaves to a chest of a human and detecting a movement of the chest from reflected waves, an image sensor for obtaining a face image of a human, and so on. By introducing such a technology, it is considered that the above-mentioned problem of burden in wearing can be solved.

However, when such a sensor is used, the human does not feel much restraint and can move freely, therefore a positional relationship between the sensor and the human, sound of surrounding environment, vibration, light, etc would change. For this reason, the noise caused by these changes is mixed with the pulse wave, it makes difficult to calculate the heart rate with high time resolution and high accuracy, and as a result, there is a problem that the state of the human cannot be estimated with high accuracy.

An example of object of the present invention is to provide a state estimation apparatus, a state estimation method, and a computer-readable recording medium that solve the aforementioned problem and estimate a state of a human with high accuracy while suppressing burden due to wearing.

### Means for Solving the Problems

In order to achieve the above object, a state estimation apparatus in one aspect of the present invention includes:

a filter generation unit configured to generate a filter that remove noise contained in a pulse wave data of a human based on a statistical value of the heart rate within a certain period of time in,

a noise removal unit configured to remove the noise contained in the pulse wave data by using the filter,

a state estimation unit configured to estimate a state of the human based on the pulse wave data after the noise removal.

Further, in order to achieve the above object, a state estimation method in one aspect of the present invention includes:

(a) a step of generating a filter that remove noise contained in a pulse wave data of a human based on a statistical value of the heart rate within a certain period of time in the pulse wave data,

(b) a step of removing the noise contained in the pulse wave data by using the filter, and

(c) a step of estimating a state of the human based on the pulse wave data after the noise removal.

Further, in order to achieve the above object, a computer-readable recording medium that includes a program recorded thereon, the program including instructions that cause a computer to carry out:

(a) a step of generating a filter that remove noise contained in a pulse wave data of a human based on a statistical value of the heart rate within a certain period of time in the pulse wave data,

(b) a step of removing the noise contained in the pulse wave data by using the filter, and

(c) a step of estimating a state of the human based on the pulse wave data after the noise removal.

### Advantageous Effects of the Invention

As described above, according to the present invention, it is possible to estimate a state of a human with high accuracy while suppressing burden due to wearing.

### BRIEF DESCRIPTION OF THE DRAWINGS

FIG. 1 is a block diagram showing a configuration of a state estimation apparatus according to the example embodiment.

FIG. 2 is a block diagram showing a configuration of a filter generation unit shown in FIG. 1 more concretely.

FIG. 3A is a diagram showing an example of a pulse wave before noise removal input to a noise removal unit shown in FIG. 1, and FIG. 3B is a diagram showing an example of a pulse wave after noise removal output from the noise removal unit shown in FIG. 1.

FIG. 4 is a flow diagram showing operation of the state estimation apparatus according to the example embodiment.

FIG. 5 is a block diagram showing an example of a computer that realizes the state estimation apparatus according to the example embodiment.

### EXAMPLE EMBODIMENTS

(Example Embodiment)

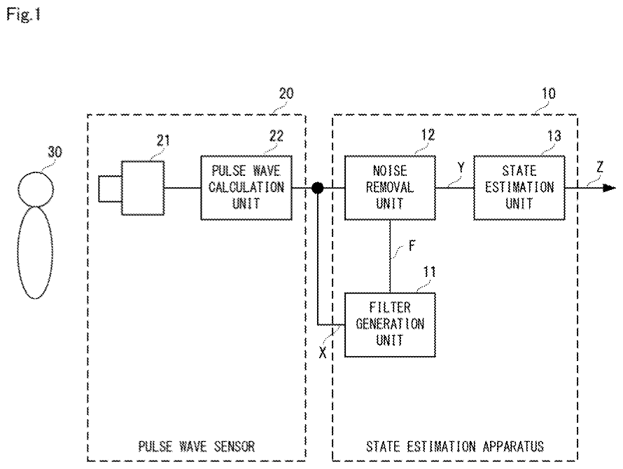

Hereinafter, a state estimation apparatus, a state estimation method, and a program according to the example embodiment will be described with reference to FIGS. 1 to 5.

[Apparatus Configuration]

First, the configuration of the state estimation apparatus according to the example embodiment will be described with reference to FIG. 1. FIG. 1 is a block diagram showing a configuration of the state estimation apparatus according to the example embodiment.

The state estimation apparatus 10 according to the example embodiment shown in FIG. 1 is an apparatus that estimates the state of the human 30. As shown in FIG. 1, the state estimation apparatus 10 includes a filter generation unit 11, a noise removal unit 12, and a state estimation unit 13.

The filter generation unit 11 generates a filter for removing noise included in a pulse wave data of a human (time-series signal of a pulse wave) based on a statistical value of a heart rate within a certain time interval in the pulse wave data. In the example embodiment, the filter generation unit 11 generates, for example, the filter for emphasizing heart rate component and heart rate variable component by removing the noise contained in the pulse wave X from the time series signal of the pulse wave X of the human 30.

The noise removal unit 12 removes the noise contained in the pulse wave data by using the generated filter. In the example embodiment, the noise removal unit 12 removes the noise contained in the pulse wave X by convolving the generated filter and the time-series signal of the pulse wave X, and outputs the pulse wave Y after removing the noise.

The state estimation unit 13 estimates a state of a human based on the pulse wave data after the noise is removed. In the example embodiment of the invention the state estimation unit 13 estimates a state of the human 30 based on the time-series signal of the pulse wave Y after removing the noise, and outputs state Z as estimation result.

As described above, in the example embodiment the filter for removing the noise included in the time-series signal of the pulse wave X is generated, and the noise included in the pulse wave X is removed by the filter. The time-series signal Y of the pulse wave after noise removal includes a heart rate component having a high time resolution included in the original pulse wave X. In addition, since the noise contained in the original pulse wave X is removed, in the time-series signal of the pulse wave Y after noise removal, a ration of the heart rate component is high, and the pulse wave Y contains a highly accurate heart rate variable component. According to the example embodiment, since the state of the human is estimated from the time-series signal of the pulse wave Y after the noise is removed, the state of the human can be estimated with high accuracy.

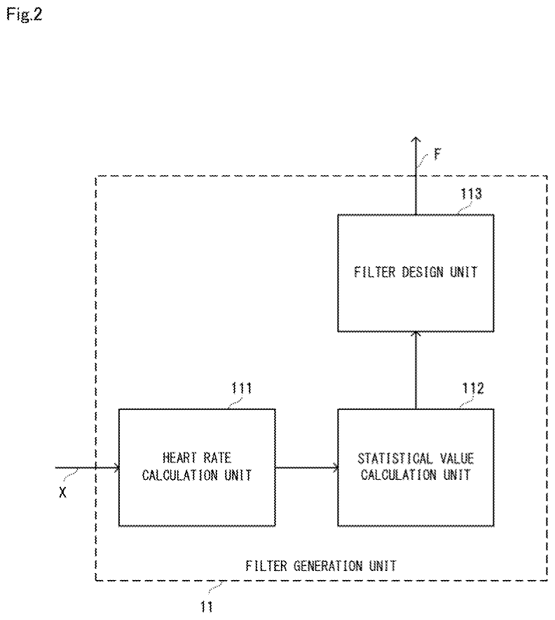

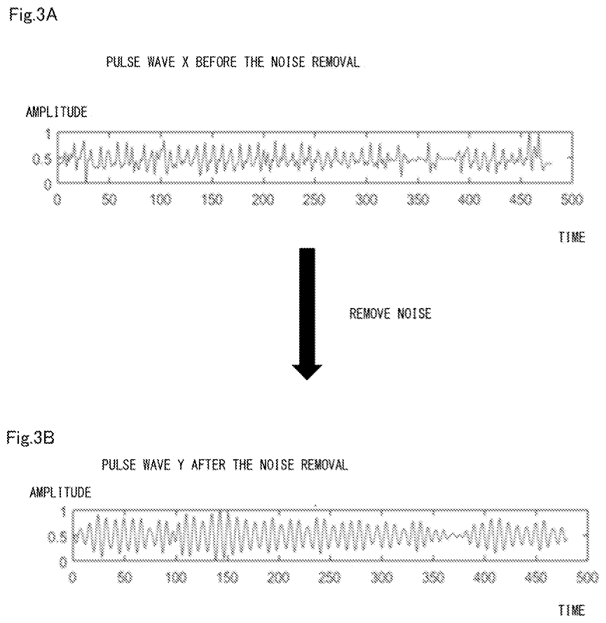

Subsequently, in addition to FIG. 1, the configuration of the state estimation apparatus 10 and the functions of each part in the example embodiment will be explained in detail by using FIGS. 2 and 3. FIG. 2 is a block diagram showing the configuration of the filter generation unit 11 shown in FIG. 1 more concretely.

As shown in FIG. 1, in the example embodiment, the state estimation apparatus 10 is connected to a pulse wave sensor 20. The pulse wave sensor 20 includes an imaging device 21 and a pulse wave calculation unit 22. The imaging device 21 is arranged so that a face of the human 30 who uses the state estimation apparatus can be photographed. The imaging device 21 outputs the image data of the captured image (image including the face) to the pulse wave calculation unit 22 at the set time interval. Example of the imaging device 21 include a digital camera, a Web camera, and the like.

In the example embodiment, the pulse wave calculation unit 22 calculates the pulse wave by the existing method, for example, by utilizing the characteristic that hemoglobin absorbs green light, and detecting the brightness (G value) of the green component in the face image.

In the above description, the pulse wave sensor 20 including the imaging device 21 and the pulse wave calculation unit 22 is explained, however the pulse wave sensor 20 is not limited to this in the example embodiment. Other pulse wave sensors 20 can be used existing pulse wave sensors such as a wearable sensor that include a wristband type wearable sensor with a built-in PPG sensor; a sensor that measures pressure and vibration installed in a bed or chair; a sensor that measures sound; and a sensor that emits microwaves to the human's chest and captures a movement of the chest from a reflected wave.

As shown in FIG. 2, in t the example embodiment, the filter generation unit 11 includes a heart rate calculation unit 111, a statistical value calculation unit 112, and a filter design unit 113.

The heart rate calculation unit 111 calculates the heart rate from the pulse wave X.

The statistical value calculation unit 112 calculates a statistical value of the heart rate within a certain period of time W0. The filter design unit 113 designs the filter that passes only the heart rate component contained in the pulse wave and removes the noise component by using the statistical value of the heart rate, and outputs designed filter F.

Specifically, the heart rate calculation unit 111 performs frequency analysis such as a fast Fourier transform on the time-series signal of the pulse wave X in a time window at a time W1 (for example, 4 seconds) shorter than the fixed time W0 (for example, 60 seconds) for calculating the heart rate statistical value. Since the heart rate component has a time cycle, the heart rate calculation unit 111 extracts a frequency power spectrum having the highest power and calculates a frequency of the frequency power spectrum as an average heart rate in time W1.

Further, the heart rate calculation unit 111 shifts the start point of the time window for frequency analysis by a time W2 (for example, 1 second) shorter than the time W1 and repeats the frequency analysis, thereby the series signal of the heart rate included in the certain period of time W0 can be obtained.

When the pulse wave X does not include noise, the heart rate can be calculated with high time resolution and high accuracy by setting the time W1 short (for example, 0.25 seconds). However, the time W1 must be set longer (for example, 10 seconds) according to the noise increases, otherwise the frequency power spectrum corresponding to the heart rate component is buried in the noise, and the heart rate cannot be calculated with high accuracy. Further, even if the time W2 is set shorter (for example, 0.25 seconds), unless the time W1 is set shorter, almost the same heart rate can be obtained, and the heart rate with high time resolution cannot be obtained. Therefore, when the pulse wave X includes noise, the heart rate cannot be calculated with high time resolution and high accuracy.

On the other hand, in generating the filter, it is sufficient to know in which range the heart rate contained in a certain period of time W0 is distributed. Therefore, the heart rate calculation unit 111 may calculate the heart rate with a low time resolution.

The statistical value calculation unit 112 calculates a statistical value indicating a range in which the heart rate in the certain period of time W0 is distributed, by using the time-series signal of the heart rate included in the certain period of time W0. For example, the statistical value calculation unit 112 calculates a maximum value and a minimum value to determine the range. Also, assuming that the time series signal of the heart rate is normally distributed, if an average value M and a standard deviation S are calculated and the range is set to M±3S, 99.7% of the data will be included in the range. Upper limit of existence range of heart rate is defined as U, and lower limit is defined as L.

The filter design unit 113 designs a bandpass filter that passes a frequency component corresponding to the lower limit L to the upper limit U of the existence range of the heart rate by an existing method, and outputs bandpass filter F. The bandpass filter F allows to pass the heart rate component contained in the pulse wave X of the certain period of time W0, however can block other noise.

The heart rate changes from moment to moment according to the state of the human 30, but the filter generation unit 11 sequentially generates a filter for the pulse wave X for a certain period of time W0 at different times. With this filter, only the heart rate component can be effectively passed, and noise can be blocked.

As shown in FIGS. 3A and 3B, the noise removal unit 12 performs a convolutional calculation between the bandpass filter F generated by the filter generation unit 11 and the time-series signal of the pulse wave X containing noise to obtains a time-series signal of pulse wave Y after noise removal. FIG. 3A is a diagram showing an example of a pulse wave before noise removal input to the noise removal unit shown in FIG. FIG. 3B is a diagram showing an example of a pulse wave after noise removal output from the noise removal unit 12 shown in FIG.

As understood from the comparison between FIGS. 3A and 3B, in the pulse wave Y after noise removal, the heart rate component which is a periodicity signal is emphasized, due to the reduction of noise as compared with the pulse wave X before noise removal. Since the bandpass filter passes frequencies in the existence range of the heart rate, the heart rate variable component contained in the pulse wave X is retained in the pulse wave Y while maintaining a high time resolution.

The state estimation unit 13 receives the time-series signal of the pulse wave Y after noise removal, which is the output of the noise removal unit 12, and outputs the state estimation value Z with reference to the state estimation model learned in advance. For example, assuming that the state in which the human 30 is awake is 1 and the state in which the human 30 is sleeping is 0, the estimation value Z is either 1 or 0. Of course, if the presence or absence of drowsiness is defined by 1 (yes) and 0 (no) and the state estimation model is learned, the presence or absence of drowsiness can be estimated (discriminated). Further, if the degree of drowsiness or stress is defined by continuous values of 1 to 0 and the state estimation model is learned, the estimation value Z becomes an estimation value of 1 to 0. In addition, if determination of emotions, for example, whether the human is frustrated, impatient, fun, happy, etc. is defined with 1 or 0 and the state estimation model is learned, it is possible to determine emotions by the estimation Z value. If defined in the same way, mental workload (how much the brain works) and the presence or absence of arrhythmia, etc. can be determined.

Examples of the state estimation model used by the state estimation unit 13 include existing neural networks such as a convolutional neural network and a recurrent neural network used in Non-Patent Document 1. In the learning of the state estimation model, i.e., the neural network, a plurality of set of the time series signal of the pulse wave containing no noise, for example, the time series signal shown in FIG. 3(b) and a state label corresponding to the time series signal (correct answer label of the state) is prepared, and a learning algorithm such as an error back propagation method is performed so that the output when the pulse wave is input to the neural network matches the state label.

### [Apparatus Operations]

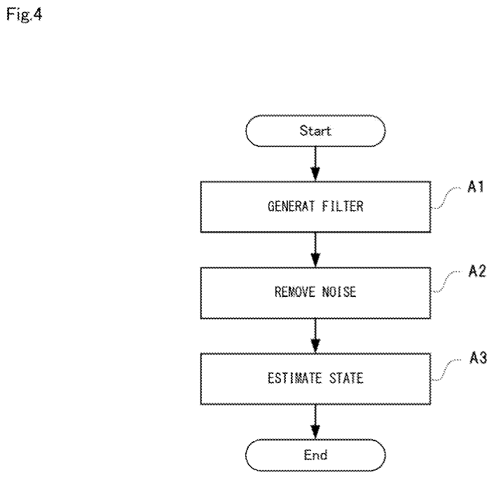

Next, the operation of the state estimation apparatus 10 in the example embodiment of the invention will be described with reference to FIG. 4. FIG. 4 is a flow diagram showing the operation of the state estimation apparatus according to the example embodiment. In the following description, FIGS. 1 to 3 will be referred as appropriate. Further, in the example embodiment, the state estimation method is implemented by operating the state estimation apparatus 10. Therefore, the description of the state estimation method in the example embodiment will be replaced with the following description of the operation of the state estimation apparatus 10.

First, as shown in FIG. 4, the filter generation unit 11 receives the time-series signal of the pulse wave X output by the pulse wave sensor 20 and generates the filter F based on the received pulse wave X (step A1).

Next, the noise removal unit 12 performs the convolutional calculation of the filter F generated in step A1 and the time series signal of the pulse wave X of the pulse wave output by the pulse wave sensor 20, to obtain the time series signal of the pulse wave Y after noise removal. (Step A2).

After that, the state estimation unit 13 estimates the state Z of the human 30 based on the time-series signal of the pulse wave Y after noise removal in step A2 and the state estimation model learned in advance (step A3).

Further, after executing step A3, when the time-series signals of the pulse waves X at different times are input to the state estimation apparatus 10, step A1 is executed again. By repeatedly executing steps A1 to A3, the state estimation apparatus 10 can continuously estimate the state of the human 30.

### [Modification Example]

The modification of the example embodiment will be described below.

In the present modification, the filter generation process in the filter generation unit 11 is performed in advance. The filter generation unit 11 generates a large number of filters F1 to FN corresponding to each of pulse waves X1 to XN from the time-series signals of each of a large number of pulse waves X1 to XN including noise prepared in advance. Then, the noise removal unit 12 obtains a large number of time-series signals of the pulse waves Y1 to YN after removing noise by applying each of the generated filters F1 to FN.

The noise removal unit 12 can learn the conversion function (corresponding to the sum of F1 to FN) that converts each of pulse waves X1 to XN into pulse waves Y1 to YN in a manner of executing a method called Denoising Auto Encoder by using a large number of pairs (X1, Y1) to (XN, YN) of pulse waves X and pulse waves Y as learning data. Assuming that the conversion function is F, the noise removal unit 12 reads the conversion function F learned in advance from the filter generation unit 11 and applies it to the time-series signal of the new pulse wave X, and thereby obtains a time-series signal of pulse wave Y after noise removal.

As described above, according to the present modification, it is not necessary to generate a filter for each time-series signal of the new pulse wave X. Therefore, there is an advantage that the time-series signal of the new pulse wave X can be efficiently processed.

### [Effect in the Example Embodiment]

As described above, according to the present embodiment, a filter for removing the noise contained in the time-series signal of the pulse wave X is generated, and the noise contained in the pulse wave X is removed by the filter. The time-series signal of the pulse wave Y after noise removal includes a heart rate component having a high time resolution included in the original pulse wave X. In addition, since the noise contained in the original pulse wave X is removed, in the time-series signal of the pulse wave Y of the pulse wave after noise removal, the ratio of the heart rate component is high, and the pulse wave Y includes a highly accurate heart rate variable component. Since the state of human is estimated from the time-series signal of the pulse wave Y after the noise is removed, the state of human can be estimated with high accuracy.

[Program]

It is sufficient for the program according to the example embodiment to be a program that causes a computer to execute steps A1 to A3 shown in FIG. 4. The state estimation apparatus 10 and the state estimation method according to the example embodiment can be realized by installing this program in the computer and executing this program. In this case, a processor of the computer functions as the filter generation unit 11, the noise removal unit 12, and the state estimation unit 13, and performs processing.

Also, the program according to the example embodiment may also be executed by a computer system constituted by a plurality of computers. In this case, for example, each computer may function as any of the filter generation unit 11, the noise removal unit 12, and the state estimation unit 13.

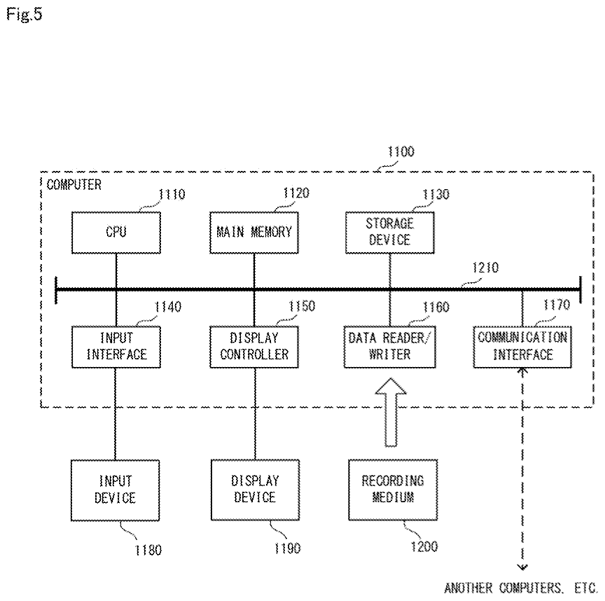

Here, a computer that realizes the state estimation apparatus 10 by executing a program of the example embodiment will be described using FIG. 5. FIG. 5 is a block diagram showing an example of a computer that realizes the state estimation apparatus according to the example embodiment.

As shown in FIG. 5, a computer 1100 includes a CPU (Central Processing Unit) 1110, a main memory 1120, a storage device 1130, an input interface 1140, a display controller 1150, a data reader/writer 1160, and a communication interface 1170. These components are connected in such a manner that they can perform data communication with one another via a bus 1210. Note that the computer 1100 may include a GPU (Graphics Processing Unit) or an FPGA (Field-Programmable Gate Array), in addition to the CPU 1110 or instead of the CPU 1110.

The CPU 1110 carries out various types of calculation by deploying the program (codes) according to the present example embodiment stored in the storage device 1130 to the main memory 1120 and executing the codes in a predetermined order. The main memory 1120 is typically a volatile storage device such as a DRAM (Dynamic Random-Access Memory). Also, the program according to the example embodiment is provided in a state of being stored in a computer-readable recording medium 1200. Note that the program according to the example embodiment may be distributed over the Internet connected via the communication interface 1170.

Also, specific examples of the storage device 1130 include a hard disk drive and a semiconductor storage device, such as a flash memory. The input interface 1140 mediates data transmission between the CPU 1110 and input device 1180 such as a keyboard and a mouse. The display controller 1150 is connected to a display device 1190 and controls display on the display device 1190.

The data reader/writer 1160 mediates data transmission between the CPU 1110 and the recording medium 1200, reads out the program from the recording medium 1200 and writes the results of processing in the computer 1100 to the recording medium 1200. The communication interface 1170 mediates data transmission between the CPU 1110 and another computer.

Also, specific examples of the recording medium 1200 include: a general-purpose semiconductor storage device, such as CF (CompactFlash®) and SD (Secure Digital); a magnetic recording medium, such as a flexible disk; and an optical recording medium, such as a CD-ROM (Compact Disk Read Only Memory).

Note that the state estimation apparatus 10 in the example embodiment can be realizable by using item of hardware that respectively corresponds to the components, rather than the computer in which the programs is installed. Furthermore, a part of the state estimation apparatus 10 may be realized by the program, and the remaining part of the state estimation apparatus 10 may be realized by hardware.

A part or an entirety of the above-described example embodiment can be represented by (Supplementary Note 1) to (Supplementary Note 3) described below, but is not limited to the description below.

(Supplementary Note 1)

A state estimation apparatus, including:

a filter generation unit configured to generate a filter that remove noise contained in a pulse wave data based on a statistical value of the heart rate within a certain period of time in the pulse wave data of a human,

a noise removal unit configured to remove the noise contained in the pulse wave data by using the filter,

a state estimation unit configured to estimate a state of the human based on the pulse wave data after the noise removal.

(Supplementary Note 2)

A state estimation method, including:

(a) a step of generating a filter that remove noise contained in a pulse wave data based on a statistical value of the heart rate within a certain period of time in the pulse wave data of a human,

(b) a step of removing the noise contained in the pulse wave data by using the filter,

(c) a step of estimating a state of the human based on the pulse wave data after the noise removal.

(Supplementary Note 3)

A computer-readable recording medium that includes a program recorded thereon, the program including instructions that cause a computer to carry out:

(a) a step of generating a filter that remove noise contained in a pulse wave data based on a statistical value of the heart rate within a certain period of time in the pulse wave data of a human,

(b) a step of removing the noise contained in the pulse wave data by using the filter,

(c) a step of estimating a state of the human based on the pulse wave data after the noise removal.

Although the present invention has been described above with reference to the example embodiments, the present invention is not limited to the above example embodiments. The configuration and details of the present invention can be made various changes that can be understood by those skilled in the art within the scope of the present invention.

### INDUSTRIAL APPLICABILITY

As described above, according to the present invention, it is possible to estimate the state of the human with high accuracy while suppressing the wearing load. The present invention is useful for various systems such as a system for estimating a human condition, for example, a vehicle driving support system, a computer system for business use, and the like.

### REFERENCE SIGNS LIST

10 state estimation apparatus

11 filter generation unit

12 noise removal unit

13 state estimation unit

20 pulse wave sensor

21 imaging device

22 pulse wave calculation unit

30 human

111 heart rate calculation unit

112 statistical value calculation unit

113 filter design unit

1100 computer

1110 CPU

1120 main memory

1130 storage device

1140 input interface

1150 display controller

1160 data reader/writer

1170 communication interface

1180 input device

1190 display device

1200 recording medium

1210 bus

What is claimed is:

1. A state estimation apparatus comprising:
a filter generation unit configured to generate a filter that remove noise contained in a pulse wave data of a human based on a statistical value of the heart rate within a certain period of time in the pulse wave data,
a noise removal unit configured to remove the noise contained in the pulse wave data by using the filter,
a state estimation unit configured to estimate a state of the human based on the pulse wave data after the noise removal.

2. A state estimation method comprising:
generating a filter that remove noise contained in a pulse wave data of a human based on a statistical value of the heart rate within a certain period of time in the pulse wave data,
removing the noise contained in the pulse wave data by using the filter,
estimating a state of the human based on the pulse wave data after the noise removal.

3. A non-transitory computer-readable recording medium that includes a program recorded thereon, the program including instructions that cause a computer to carry out:
generating a filter that remove noise contained in a pulse wave of a human data based on a statistical value of the heart rate within a certain period of time in the pulse wave data,
removing the noise contained in the pulse wave data by using the filter,
estimating a state of the human based on the pulse wave data after the noise removal.
